(19) United States
(12) Patent Application Publication
(10) Pub. No.: US 2017/0313973 A1
(43) Pub. Date: Nov. 2, 2017

(54) USE OF BACTERIAL BIOFILMS IN OENOLOGY

(71) Applicants: UNIVERSITE DE BOURGOGNE, Dijon (FR); INSTITUT NATIONAL SUPERIEUR DES SCIENCES AGRONOMIQ AGRONOMIQUES, DE L'ALIMENTATION ET DE, Dijon (FR)

(72) Inventors: Jean GUZZO, Fontaine les Dijon (FR); Stéphanie WEIDMANN-DESROCHE, Quetigny (FR); Alexandre BASTARD, Dijon (FR); Christian COELHO, Entraigues (FR); Régis GOUGEON, Quetigny (FR)

(73) Assignees: Universite de Bourgogne, Dijon (FR); Institut National Superieur des Sciences Agronomiques, de L'Alimentation et de, Dijon (FR); L'Environnement, Dijon (FR)

(21) Appl. No.: 15/517,691

(22) PCT Filed: Oct. 9, 2015
(86) PCT No.: PCT/EP2015/073343

§ 371 Date: Apr. 7, 2017

(30) Foreign Application Priority Data
Oct. 10, 2014 (FR) 1459736

(51) Int. Cl. C12N 1/16; C12P 7/14; C12N 1/22; C12P 7/12; C12P 7/00; C12N 1/00

(52) U.S. Cl. CPC C12N 1/16; C12N 1/22; C12P 7/12; C12P 7/14; C12N 1/00; C12P 7/00

(57) ABSTRACT

A method for preparing a fermented drink is described. The method comprises the initiation of fermentation by inoculating the fermentable drink with fermentative bacteria in the form of biofilm.

### FIELD OF THE INVENTION

The present invention relates to a method for preparing a fermented drink comprising the initiation of fermentation by inoculating the fermentable drink with fermentative bacteria, and optionally fermentative yeasts, in the form of biofilm. In particular, the present invention relates to a method for preparing wines comprising the initiation of malolactic fermentation by inoculation with lactic bacteria in the form of biofilm.

### PRIOR ART

Nowadays, in the modern oenology field, control of the different steps of the elaboration of wine requires the use of yeast ferments for alcoholic fermentation and bacterial ferments for malolactic fermentation. Malolactic fermentation in general takes place after alcoholic fermentation. It consists in a transformation of malic acid into lactic acid by means of bacteria called lactic bacteria. Malolactic fermentation results in a reduction in acidity, enabling a stabilisation and a softening of the wine, as well as maintaining constant quality from one year to the next.

Since malolactic fermentation influences the organoleptic qualities of wines, it has to be strictly controlled. Since spontaneous fermentation can be initiated at any moment of the process, means for initiating this fermentation at the right time have been developed. Thus, so-called “ready-to-use” lactic bacteria have been developed. These bacteria make it possible to improve the control of conducting malolactic fermentation and to produce wines of constant quality with a certain typicity meeting consumer demand. Oenococcus oeni is the bacterial species the most frequently used and the best suited for carrying out malolactic fermentation. Thus, compared to random malolactic fermentation carried out with indigenous lactic bacteria, bacterial seeding has numerous advantages.

However, since the physical-chemical conditions of the wine (acid pH, high ethanol content, presence of sulphite, low temperature, nutritional deficiency) are unfavourable to the implantation of these lactic ferments during inoculation, it has been necessary to develop industrial protocols for pre-acclimatisation to the conditions of the wine to overcome these problems so as to optimise the survival and the resumption of growth of the bacteria in the wine after direct inoculation. In particular, the Lallemand S.A.S. company uses pre-acclimatisation protocols called MBR® or 1-STEP®. The malolactic fermentation is often carried out in barrels. The malolactic ferments are introduced therein in frozen or lyophilised form and develop in planktonic form. The bacterial strains thus acclimatised remain expensive for the wine producer.

A need thus remains for the development of an easy to implement method making it possible to initiate fermentation, in particular malolactic fermentation, in a reproducible manner and at lower cost. Advantageously, the method will make it possible to modulate the organoleptic properties of the fermented drinks thus prepared.

### BRIEF DESCRIPTION OF THE INVENTION

The present invention pertains to a method for preparing a fermented drink which comprises the initiation of fermentation by inoculating the fermentable drink with fermentative bacteria in the form of biofilm.

The present invention also pertains to a fermented drink capable of being obtained by such a method.

### BRIEF DESCRIPTION OF THE FIGURES

FIG. 1 represents the survival of cells of O. oeni as a function of time in a growth medium (MRSm) modified by addition of ethanol and lowering of the pH to 3.0 in order to generate stressful conditions.

FIG. 2A represents the malolactic activity of cells of O. oeni cultured beforehand either in biofilm (curve with squares) or in planktonic culture (curve with circles) and incubated in a standardised fermented grape juice with 12% of ethanol, 4 g/l of L-malic acid, pH 3.5.

FIG. 2B represents the malolactic activity of cells of O. oeni cultured beforehand either in biofilm (curve with squares) or in planktonic culture (curve with circles) and incubated in a standardised fermented grape juice with 12% of ethanol, 4 g/l of L-malic acid, pH 3.2.

FIG. 3 represents the assays (concentration in μg/l) of 6 marker aromas of wood (from top left to bottom right: furfural, vanillin, guaiacol, eugenol, cis-whisky lactone, trans-whisky lactone) in wines of different conditions:

Left bar: “Oak” (wood-wine contact, without microbiological activity),

Central bar: “FML+oak” (malolactic fermentation by planktonic bacteria during the wood-wine contact time),

Right bar: “Biofilm” (biofilm formed on oak wood, carrying out the malolactic fermentation, releasing wood aromas into the wine).

Significant differences (P=0.05) between the conditions are mentioned by different letters.

FIG. 4 represents the principal component analysis (84%) of the projection of the set of assay data of the 6 marker aromas of the wood in wines of different conditions: “Oak” (wood-wine contact, without microbiological activity), “FML+oak” (malolactic fermentation carried out by planktonic bacteria during the wood-wine contact time), “Biofilm” (biofilm formed on oak wood, carrying out the malolactic fermentation, releasing wood aromas into the wine).

FIG. 5 represents the measurement of colour by the parameters L* a* b* (respectively white, black and grey histograms) of four wines of different conditions (from left to right):

“Wine” (initial wine before experimentation),

“Oak” (wood-wine contact, without microbiological activity),

“FML+oak” (malolactic fermentation carried out by planktonic bacteria during the wood-wine contact time),

“Biofilm” (biofilm formed on oak wood, carrying out the malolactic fermentation, releasing wood aromas into the wine).

Significant differences (P=0.05) between the conditions are mentioned by different letters.

### DETAILED DESCRIPTION OF THE INVENTION

The inventors have demonstrated that the inoculation of fermentable drinks with fermentative bacteria in the form of biofilm makes it possible to initiate fermentation in a reproducible manner, at lower cost and without implementation difficulties. In particular, the inventors have demonstrated that the inoculation of fermentable drinks with lactic bacteria in the form of biofilm makes it possible to initiate malolactic fermentation in a reproducible manner, at lower cost and without implementation difficulties. Advantageously, in certain embodiments, the inoculation of fermentable drinks with lactic bacteria in the form of biofilm makes it possible to modulate the organoleptic properties of the fermented drinks thus prepared.

The present invention thus pertains to a method for preparing a fermented drink comprising the initiation of fermentation by inoculating the fermentable drink with fermentative bacteria in the form of biofilm. In particular, the present invention pertains to a method for preparing a fermented drink comprising the initiation of malolactic fermentation by inoculating the fermentable drink with lactic bacteria in the form of biofilm.

The fermented drink may be any fermented drink. In particular, the fermented drink may be selected from wines, such as red wines, white wines, rosé wines and champagnes, fruit and/or vegetable based drinks, vinegars, ciders or beers. Preferably, the fermented drink is a wine.

The fermentative bacteria may be selected from lactic bacteria, acetic bacteria and combinations thereof. In particular, the fermentative bacteria may be selected from bacterial strains belonging to the genus Oenococcus, Lactobacillus, Pediococcus, Weissella, Leuconostoc or combinations thereof. Quite particularly, the fermentative bacteria are lactic bacteria, preferably Oenococcus oeni.

In certain embodiments, the method of the present invention comprises the inoculation of the fermentable drink with a combination of fermentative bacteria and fermentative yeasts in the form of biofilm. It should be understood that the biofilm comprises the bacteria and the fermentative yeasts in combination. Such a biofilm is known as “mixed”. The fermentative bacteria may be as described above. The yeasts may be selected from strains belonging to the genus Saccharomyces, Schizosaccharomyces, Brettanomyces, Torulaspora, Candida, Metschnikowia, Kluyveromyces or combinations thereof. Co-inoculation advantageously makes it possible to initiate the sequence of alcoholic fermentation and malolactic fermentation. It can also make it possible to develop the flavours of the fermented drink produced.

Thus, in the method of the present invention, fermentation may be initiated by inoculating the fermentable drink with fermentative bacteria, which may be of the same strain or be a combination of two or more strains of the same species or instead be a combination of different bacterial species, in the form of biofilm, or be initiated by inoculation with bacteria and fermentative yeasts, the bacteria and the yeasts being in combination in the form of a biofilm. Such biofilms are known as “mixed biofilms” (combinations of different bacterial species or combinations of bacteria and yeast).

The fermentative bacteria in the form of biofilm, as well as yeasts when present, have the advantage of being more resistant to stresses (low pH, high ethanol content; for example for wine, pH below 3.5, concentration of ethanol greater than or equal to 12% by volume, etc.) compared to bacteria, and if need be yeasts, not organised in such a way. It thus appears no longer necessary to pre-acclimatise the bacteria to the physical-chemical conditions of the drinks to ferment, which makes it possible to reduce considerably the costs linked to the preparation of bacteria, optionally in combination with “ready-to-use” yeasts.

Biofilms are three-dimensional structures constituted of bacterial populations incorporated in an exopolymeric matrix (polysaccharides, polypeptides, nucleic acids). They develop on different types of support. Often considered as harmful due to the fact that they can constitute a reserve of micro-organisms and be the source of recurrent contaminations of the surrounding medium, biofilms have until now been slightly valued.

Generally speaking, the formation of biofilms results from a dynamic and complex process. The biofilm is constituted in several steps from the initial adhesion to a support to the formation of a three-dimensional structure which can constitute a genuine ecosystem assuring various functions. The exopolymeric matrix is a key element of the biofilm and constitutes a genuine physical and biological barrier which protects the bacteria from environmental stresses (antimicrobial agents, acid stresses, thermal stresses, etc.) but also plays the role of filter by supplying the biofilm with nutrients. The bacteria present in the biofilm benefit from this favourable environment to proliferate and develop a “Biofilm” phenotype, which confers them with a very high resistance to stress and specific functionalities (Rieu et al., 2014).

The biofilm may be prepared by incubating micro-organisms in a medium favourable to their growth in the presence of the solid support. The growth time may depend on the species and, within a species, the strain used. For example, in the case of the strain O. oeni ATTCC BBA-1163, the incubation lasts at the most 7 days in modified MRS medium.

The inventors have shown that fermentative bacteria in the form of biofilm, in particular cells of O. oeni cultured in biofilm, have increased resistance to stress (low pH, high ethanol content; for example for wine, pH below 3.5, ethanol concentration greater than or equal to 12% by volume, etc.) compared to cells cultured in liquid medium. This increased stress resistance enables them to withstand the unfavourable physical-chemical conditions which can be encountered during the preparation of fermented drinks, in particular wine.

Furthermore, the malolactic performances of the bacteria in the form of biofilm are greater than those of bacteria commonly grown in planktonic culture.

The phenotype acquired by the bacteria during growth in biofilm confers on them this resistance, which ends up in better performances for using malate and thus carrying out malolactic fermentation of wine in a shortened time.

Thus, bacteria and yeasts in the form of biofilm have enhanced stress resistance. Furthermore, the constituents of the exopolymeric matrix of the biofilm may advantageously make it possible to modulate the organoleptic qualities and flavours of the prepared fermented drink. Thus, the method of the present invention may enable the preparation and the modulation of the organoleptic properties of a fermented drink by inoculating the fermentable drink with fermentative bacteria, and potentially yeasts, in the form of biofilm. In particular, the method of the present invention may make it possible to modulate the transfer of aromatic molecules from the wood to the fermented drink and/or to modulate the transfer of pigments from the wood to the fermented drink.

Since the constituents of the exopolymeric matrix of the biofilm are able, per se, to modulate the organoleptic qualities of drinks (fermented or not), they may be used, after extraction of the biofilm, in methods for modulating the organoleptic qualities of drinks. Thus, the present invention also pertains to the use of constituents, in particular exopolysaccharides, of the exopolymeric matrix of a biofilm of bacteria, and potentially of yeasts, for modulating the organoleptic qualities of a drink.

The fermentative bacteria in the form of biofilm, or combinations of bacteria and fermentative yeasts in the form of biofilm, may be inoculated in a form removed from the support, that is to say after removal from the support having enabled the development of the biofilm, or in a form adherent to the support, that is to say still attached to the support having enabled the development of the biofilm. The support may be any suitable abioitic support. In particular, the support may be selected from wood, quite particularly selected from species of wood used as containing fermented liquid product, cork, stainless steel, polystyrene, silicone or polyethylene composites. The wood support could be in different forms, for example wood chips. When the support is of the wood, it may be heated or non-heated. The support is typically in the form of forestry chips (parallelepipeds) or granulates.

The fermentative bacteria in the form of biofilm or combinations of bacteria and fermentative yeasts in the form of biofilm, removed from or adherent to the support, may be in different forms which maintain their viability and vitality, such as under vacuum, frozen or lyophilised.

The fermentative bacteria, or combinations of bacteria and fermentative yeasts removed from the biofilm may, in particular, be in frozen form or in lyophilised form.

The fermentative bacteria, or combinations of bacteria and fermentative yeasts in biofilm (adherent) may be in frozen form, in lyophilised form, under vacuum, under controlled atmosphere or instead in native form.

The purified exopolymeric matrix of the biofilm may be in frozen form, lyophilised, under vacuum, under inert atmosphere, or in native form and may be added to develop the organoleptic qualities of fermented or non-fermented drinks.

Those skilled in the art will know how to determine the quantity of fermentative bacteria that has to be inoculated in order to initiate fermentation, this quantity varying as a function of the nature of the drink to ferment and the fermentative bacteria chosen.

Typically, a minimum of 10⁶ to 10⁷ CFU/mL is employed to initiate malolactic fermentation during the preparation of wines. Advantageously, when the support is wood and when the bacteria, potentially in combination with yeasts, are inoculated in a form adherent to the support, the inoculation may make it possible to modulate the organoleptic properties of the prepared fermented drinks.

It is well known that during ageing of wine in barrels, transfers take place from the wood to the wine but also from the wine to the wood. The quantities and the compounds that migrate depend on the characteristics of the wood (species, drying conditions, thermal treatment, number of uses, etc. and of the wine (pH, types of vine used, yeasts, bacteria, chemical composition, etc.). The extraction of wood compounds by wine is the subject of numerous research. Better understanding of this phenomenon enables the cellar master to determine the duration of maturation in barrels. The simple extraction of aromatic compounds (volatiles and polyphenols) and tannins from the wood adds richness and complexity to the aromas of wines (Díaz-Plaza et al., 2002, Perez-Prieto et al., 2002; Fernandez de Simón et al., 2003). A large variety of aromatic compounds belonging to different chemical families has been identified in oak wood (Cadahía et al., 2003; Fernandez de Simón et al., 2006). Ellagitannins (wood tannins) have an important antioxidant role, and thanks to their ability to consume large quantities of oxygen they are going to regulate the method of oxidation of the wine. The extraction of the principal volatile compounds stemming from the degradation of wood components during maturation and heating may increase the aromatic complexity of barrel aged wines (Cadahía et al., 2003; Fernandez de Simón et al., 2006). After their extraction, these compounds undergo a series of biochemical modifications that have an impact on the sensory properties of the wine.

TABLE 1
Odour descriptors of different wood extracts

| Compounds of oak wood | Olfactory descriptors |
|---|---|
| Whisky lactone | Vanillin, woody, clove, coconut |
| Furfural | Slightly roasted, caramel |
| 5-methylfurfural | Spicy, roasted, sweet |
| Gaiacol | Spicy, roasted, smoked/burnt |
| Eugenol | Spicy, clove, cinnamon |
| Isoeugenol | Spicy, clove, woody |
| Vanillin | Sweet, vanilla |

Wood chips and “staves” (the curved pieces of woods forming the sides of barrels) are alternatives to the use of barrels but they are not allowed by all legislations. Wood chips are mainly used to accelerate the elaboration of wine by the acquisition of woody notes because this format increases the exchange surfaces, enabling more rapid and more important transfers for the same mass of wood (Fernández de Simón et al., 2010).

The inoculation of bacteria in a form adherent to a wood support thus makes it possible to develop the organoleptic properties of the prepared drinks, in particular wines, in the manner of wood chips used as an alternative to the use of barrels.

The present invention also pertains to a fermented drink capable of being obtained by the method of the present invention as described above. Such drinks may have distinctive organoleptic properties.

### Examples

Bacterial Strain

For all the experimentations the strain used is Oenococcus oeni ATTCC BBA-1163.

The Culture Media for the Bacteria Used in this Study are the Following:

MRSm: 52 g/l MRS (Conda) to which is added: 10 g/l fructose; 8 g/l (D-L) malic acid. The pH is adjusted to 4.8.

MRSstress: MRSm to which is added 13% of ethanol, pH adjusted to 3.0.

Wine: a commercial white grape juice is fermented using an ADY (active dry yeast) then its parameters are standardised: pH to 3.2 or 3.5; 4 g/l of L-malic acid; ethanol 12% by volume. The wine is then filtered at 0.22 μm.

1. Survival of Cells in Acid Stress Condition and in the Presence of Ethanol as a Function of the Biofilm or Planktonic Culture Conditions.

The planktonic cells come from a culture of 24 h in MRSm at 28° C. The biofilm is obtained by a culture of 1 week in MRSm on steel coupon (2.5*2.5 cm) at 28° C. The sessile cells (biofilm) are mechanically removed from the steel and incubated in stress conditions (MRSstress medium). The living and growable cells are counted on a MRSm agar after 0, 1, 4 and 24 h of incubation in MRSstress.

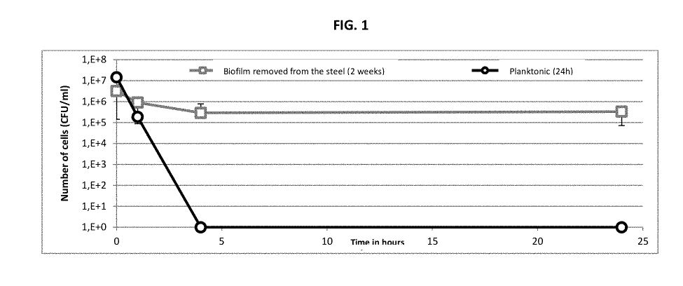

The results are presented in FIG. 1. Squares: survival kinetic of planktonic cells; circles: survival kinetic of cells removed from a biofilm on steel (2 weeks). The incubation takes place in MRSstress (13% of ethanol, pH 3).

The stress conditions used in this experiment correspond to an extremely stressful environment for the bacterium, which may correspond to the conditions present in certain wines. The planktonic cells seeded at 10⁷ CFU/ml in this medium do not survive, with total mortality after 4 hours. The cells cultured beforehand in biofilm have a log loss after 4 hours of incubation. In addition, the viability is maintained constant over 24 h.

This first experiment shows that cells of O. oeni cultured in biofilm acquire a stress resistance which confers on them the ability to survive stresses encountered in wine, these stress conditions being normally lethal for cells cultured in liquid (planktonic) medium.

2. Monitoring of Malolactic Fermentation Carried Out with O. oeni Cells Grown in Biofilm or in Planktonic Culture.

The planktonic cells are cultured in MRSm at 28° C. The biofilm is obtained by a culture of 7 days in MRSm on wood (2.5*2.5 cm). In the case of biofilms, the medium is renewed once at mid-culture time (3.5 d). Maiolactic fermentation is implemented by direct seeding of the planktonic culture (8·10⁷ CFU/ml) in 20 ml of wine or immersion of the biofilm on wood coupon (5·10⁷ CFU/ml) in the same volume.

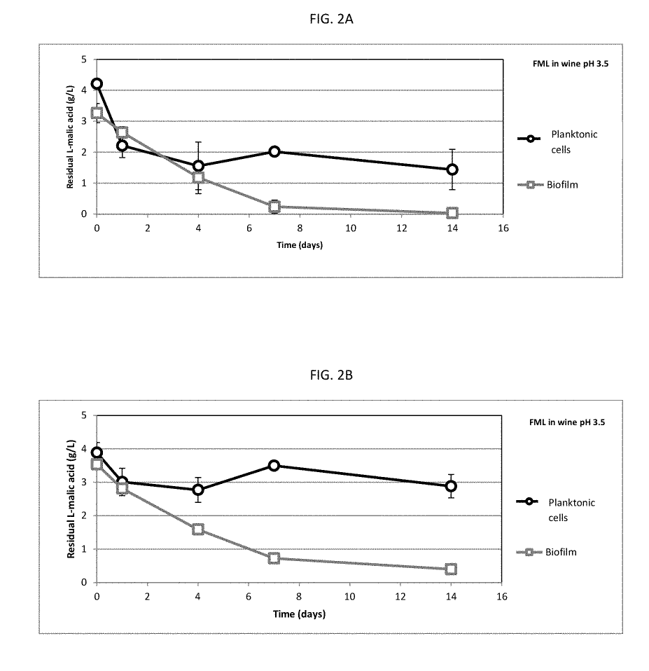

The results are presented in FIGS. 2A and 2B.

The conditions used here are representative of the wine medium. At pH 3.5, the decarboxylation of malic acid by planktonic cells is effective but incomplete with, at the end of 14 days, a residual concentration of malic acid of around 1.5 g/l. For the synthetic wine at pH 3.2, the conditions appear too drastic and the low fermentative capacity does not make it possible to consume malic acid with a residual concentration at the end of fermentation of 3 g/l. Counts of these bacteria at different incubation times made it possible to show complete mortality of the cells at the end of 4 days, which explains the stoppage of the consumption of malate from this time for planktonic cells.

On the other hand, the cells of O. oeni cultured on wood coupon are capable of finishing malolactic fermentation (complete consumption of malate) in 7 days for the synthetic wine at pH 3.5 and in 14 days for the synthetic wine at pH 3.2. The degradation kinetic is rapid and efficient and bears witness to the very good metabolic activity of the bacteria in biofilm on wood coupon.

This experiment makes it possible to conclude that the bacterium O. oeni cultured in biofilm on wood coupon is metabolically active and capable of carrying out malolactic fermentation in conditions mimicking wine and in the presence of stress. It should here be retained that:
the culture in biofilm does not require pre-acclimatisation of the bacteria to the stress conditions.
the malolactic performances of these bacteria in biofilm are greater than those of bacteria commonly grown in planktonic culture.

It is the phenotype acquired by the bacteria during growth in biofilm which confers this resistance, which ends up in better performances for using malate and thus carrying out malolactic fermentation of wine in a shortened time.

3. Influence of Biofilm in Wood-Wine Exchanges:
Physical-chemical stress conditions: pH<3.5; Ethanol 12% by volume
Organoleptic quality results:

To evaluate the influence of biofilms in wood-wine exchanges, 3 conditions were compared: (i) oak coupons immersed in the wine, designated “oak”, (ii) malolactic fermentation of wine carried out by planktonic bacteria in the presence of oak coupons, designated “FML+oak”, (iii) a biofilm of O. oeni on oak coupons capable of carrying out the FML of the wine, designated “biofilm”.

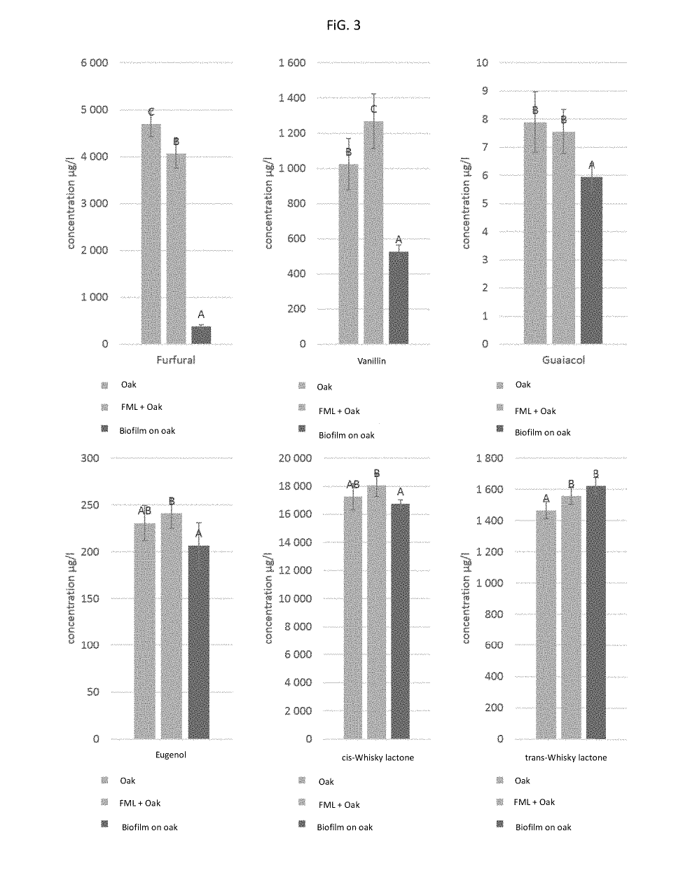

At the end of one month of maturation (contact between the wood and the wine) six aromatic molecules characteristic of the wood were assayed in the wines produced in the above 3 conditions; the aromatic molecules are furfural (almond), vanillin (vanilla), guaiacol (smoky), eugenol (clove) and cis- and trans-whisky lactones (coconut) (FIG. 3).

Three biological repetitions and two techniques were carried out.

An ANOVA statistical test followed by the Tukey HSD post hoc test were carried out on the data to determine significant differences between groups (the differences are represented by different letters: P=0.05)

The biofilm condition is different from the two other conditions for furfural, vanillin and guaiacol. The biofilm has reduced the extraction of these three aromas in the wine.

Similarly, the “biofilm” condition gives a lower concentration of eugenol and cis-whisky lactone than the “FML+oak” condition.

The “biofilm” condition improves the extraction of trans-whisky lactone compared to the “Oak” condition without microbiological activity, but is not significantly different from the “FML+oak” condition.

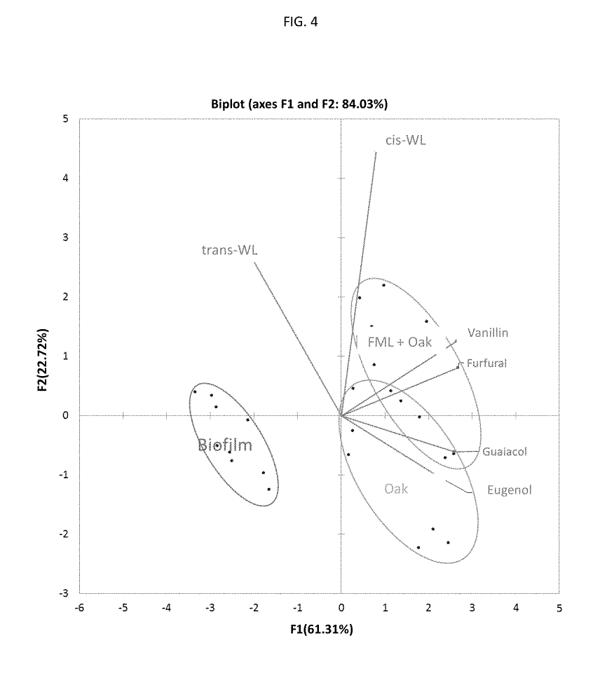

Principal component analysis of the set of data of assays of aromas shows that 84% of the variability is taken into account by the two components (FIG. 4). There is a strong correlation of the first component (61.31%) with vanillin, furfural, guaiacol and eugenol. Trans and cis-whisky lactones are strongly correlated with the component 2 (22.72%). The separation of the data observed shows that the data of the “Biofilm” condition are isolated from the data of the “Oak” and “FML+oak” conditions by the component 1, which represents 61.31% of the variability. There is thus a strong statistical difference between these conditions.

The biofilm has thus modulated the transfer of aromatic molecules from the wood to the wine.

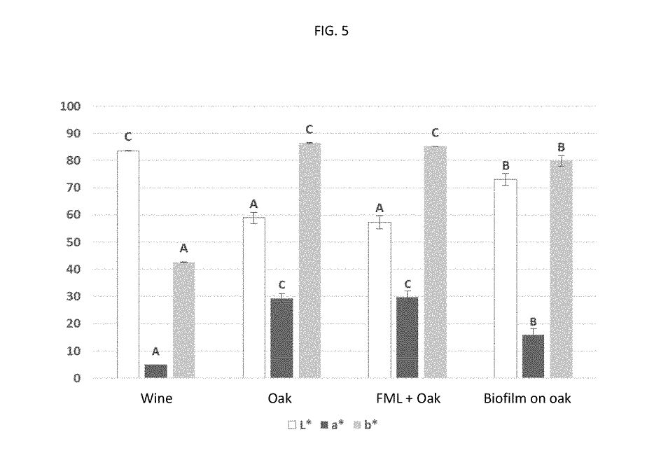

The colour of the wine of these same samples was analysed by spectrophotometry (L* a* b* parameters) (FIG. 5). The initial white wine becomes coloured through contact with the wood, which is shown by the increase in the parameters a* (red) and b* (yellow) and the reduction of L* (clarity) between “wine” and “oak”. The fermentative activity of planktonic bacteria in “FML+oak” does not significantly modify these parameters.

On the other hand, “biofilm on oak” limits coloration, the wine obtained is less orange, (a* and b*) and loses less clarity (L*).

The biofilm has thus modulated the transfer of pigments from the wood to the wine.

### BIBLIOGRAPHICAL REFERENCES

Cadahía, E., Fernández de Simón, B., and Jalocha, J. (2003). Volatile Compounds in Spanish, French, and American Oak Woods after Natural Seasoning and Toasting. J. Agric. Food Chem. 51, 5923-5932.
Díaz-Plaza, E M., Reyero, J R., Pardo, F., Alonso, G L., Salinas, M R. (2002). Influence of Oak Wood on the Aromatic Composition and Quality of Wines with Different Tannin Contents. Journal of Agricultural and Food Chemistry 50 (9), 2622-2626
Fernandez de Simón B., Hernández, T., Cadahía, E., Dueñas, M., and Estrella, I. (2003). Phenolic compounds in a Spanish red wine aged in barrels made of Spanish, French and American oak wood. Eur Food Res Technol 216, 150-156.
Fernández de Simón, B., Sanz, M., Cadahía, E., Poveda, P., and Broto, M. (2006). Chemical Characterization of Oak Heartwood from Spanish Forests of Quercus pyrenaica (Wild.). Ellagitannins, Low Molecular Weight Phenolic, and Volatile Compounds. J. Agric. Food Chem. 54, 8314-8321.

Fernandez de Simon, B., Cadahia, E., Muino, I., Del Alamo, M., & Nevares, I. 2010. Volatile Composition of Toasted Oak Chips and Staves and of Red Wine Aged with Them. American Journal of Enology and Viticulture, 61(2), 157-165.
Nel, H. A., Bauer, R., Wolfaardt, G. M., and Dicks, L. M. T. (2002). Effect of bacteriocins pediocin PD-1, plantaricin 423, and nisin on biofilms of Oenococcus oeni on a stainless steel surface. American Journal of Enology and Viticulture 53, 191-196.
Pérez-Prieto, L. J., López-Roca, J. M., Martínez-Cutillas, A., Pardo Minguez, F., and Gómez-Plaza, E. (2002). Maturing Wines in Oak Barrels. Effects of Origin, Volume, and Age of the Barrel on the Wine Volatile Composition. J. Agric. Food Chem. 50, 3272-3276.
Rieu, A., Aoudia, N., Jego, G., Chluba, J., Yousfi, N., Briandet, R., Deschamps, J., Gasquet, B., Monedero, V., Garrido, C., Guzzo Jean. (2014). The biofilm mode of life boosts the anti-inflammatory properties of Lactobacillus. Cell. Microbiol.

1-11. (canceled)

12. A method for preparing a fermented drink comprising the initiation of fermentation by inoculating the fermentable drink with fermentative bacteria in the form of biofilm.

13. The method according to claim 12 wherein the fermentable drink is moreover inoculated with fermentative yeasts, the bacteria and fermentative yeasts being in combination in the form of a biofilm.

14. The method according to claim 12 wherein the fermentation is a malolactic fermentation and the fermentative bacteria are lactic bacteria in the form of biofilm.

15. The method according to claim 12 wherein the fermented drink is selected from wines, fruit and vegetable based drinks, vinegars, ciders or beers, preferably the fermented drink is a wine.

16. The method according to claim 12 wherein the fermentative bacteria are selected from bacterial strains belonging to the genus Oenococcus, Lactobacillus, Pediococcus, Weissella, Leuconostoc or combinations thereof, preferably the fermentative bacteria are Oenococcus oeni.

17. The method according to claim 13 wherein the fermentative yeasts are selected from strains belonging to the genus Saccharomyces, Schizosaccharomyces, Brettanomyces, Torulaspora, Candida, Metschnikowia, Kluyveromyces or combinations thereof.

18. The method according to claim 12 wherein the fermentative bacteria in the form of biofilm are inoculated in a form removed from the support, or in a form adherent to the support.

19. The method according to claim 18 wherein the support is selected from wood, preferably species of wood used as containing fermented liquid product, cork, stainless steel, polystyrene, silicone or polyethylene composites.

20. The method according to claim 18 wherein the fermentative bacteria in the form of biofilm, before inoculation, are in frozen form, under vacuum or in lyophilised form.

21. The method according to claim 12 wherein the quantity of bacteria inoculated varies from 10⁶ to 10⁷ CFU/mL.

22. Fermented drinks capable of being obtained by a method according to claim 12.

23. The method according to claim 13 wherein the combination of bacteria and fermentative yeasts in the form of biofilm are inoculated in a form removed from the support, or in a form adherent to the support.

24. The method according to claim 13 wherein the combination of bacteria and fermentative yeasts in the form of biofilm, before inoculation, are in frozen form, under vacuum or in lyophilised form.
